(19) United States
(12) Patent Application Publication
(10) Pub. No.: US 2005/0196828 A1
(43) Pub. Date: Sep. 8, 2005

(54) Bioreactor with expandable surface area for culturing cells

(76) Inventors: Ming Hao Zheng, City Beach (AU); Bruno Giannetti, Bonn (DE)

Correspondence Address: MORGAN, LEWIS & BOCKIUS LLP, 1701 Market Street, Philadelphia, PA, 19103, US

(21) Appl. No.: 11/065,488

(22) Filed: Feb. 24, 2005

Related U.S. Application Data
(63) Continuation of application No. PCT/EP03/09475, filed on Aug. 27, 2003.
(60) Provisional application No. 60/406,224, filed on Aug. 27, 2002.

(51) Int. Cl. C12P 35/02

(52) U.S. Cl. 435/51

(57) ABSTRACT

The present invention discloses a bioreactor apparatus with an expandable surface area which is useful for culturing cells. A method for culturing cells is also included in the invention. The method comprises providing a container and a carrier adjacent the container, wherein the container includes an inflow and an outflow, and wherein the carrier includes an expandable surface area; supplying cells through an inflow to the carrier, wherein said cells will adhere to the carrier and proliferate; and continually supplying nutrients through the inflow of the container. A device for culturing cells comprising a carrier including an expandable surface area upon which to culture the cells is also included in the present invention.

### FIELD OF THE INVENTION

The present invention relates to the field of cell culturing techniques. In particular, the invention is directed to new methods, apparatus, and devices for culturing cells.

### BACKGROUND OF THE INVENTION

Biological products, for example monoclonal antibodies, interferers, vaccines, therapeutic proteins, tissue structures and the like, are produced with the help of cells and especially of mammalian cells. The economics of producing such products depend in part upon the efficiency of cells multiplying and upon the highest possible concentration of the substance being produced in the interior of the cells. In order to achieve this, special requirements are demanded of the processes and devices used for large scale culturing of cells. It is important to provide a sufficient supply of nutrients, as well as of gases, particularly oxygen. Waste products, for example, cell metabolic products, which are not needed in the cell culture and which frequently inhibit growth of the cells, must be removed. All other environmental conditions, such as temperature and pH value, must also be controlled.

Many animal cells used for the production of viral vaccines, growth factors, receptors or therapeutic proteins are anchorage-dependent, meaning that they must adhere to a compatible surface in order to grow. This requirement is particularly stringent for normal diploid cells; these cells not only need to attach to a surface, they develop a polarized, elongated cell shape after attachment and eventually grow to cover the surface and reach a state of confluence. These types of cells form a monolayer on a surface, and at confluence, that is, when the cells have reached a maximum density, cell division stops.

Contact-inhibition between the cultured cells also halts cell proliferation. Proliferation resumes only after the cells are detached from the surface on which the adhere by exposure to a proteolytic agent, such as trypsin, and re-plated onto a larger surface.

Conventionally, cells have been cultivated in roller bottles or on tissue culture plates or flasks, all of which have fixed surface areas. Since the 1980's, the demand for large quantities of therapeutic proteins has resulted in a wider application of many alternative cultivation methods which are more suitable for large-scale operations. These alternative methods include using bioreactors to precisely control the environmental conditions, such as pH, providing nutrients, and removing waste. For adherent cells, microcarriers can be used as an adherent surface within the bioreactor. Such microcarriers can be prepared from dextran (See Van Wezel, A. L. 1967, Nature 216:64:65); polystyrene (See Johansson, A. et al., 1980, Dev. Biol. Stand 46:125-129 and Kno, M. et al., 1981, In Vitro 17:901-906); cellulose (See Reuveny, S., et al., 1982, Dev. Biol. Stand. 50:115-123); collagen (See R. C. Dean et al., 1985, Large Scale Mammalian Cell Culture Technology. Ed. B. K. Lydersen, Hansen Publishers, New York, N.Y., pp. 145-167); or gelatin-based macroporous beads (See Cultisphere, Technical Bulletin, Percell Biolytics AB). A stirring means moves the culture medium in the interior of the reactor and thus provides a homogeneous distribution of microcarriers on which adherent cells can adhere and proliferate. Alternative bioreactors include hollow-fiber bioreactors (See Knazek, et al., 1972, Science 178:65) and ceramic bioreactors (See Lydersen, et al., 1985, Biotechnology 3:63).

Among these alternative choices to traditional cell culturing, typically bioreactors containing microcarriers are the most widely employed for adherent cells. A wide range of dimensions of the microcarrier are suitable for growth of mammalian cells, usually from 100 to 400 microns. (See e.g., Butler, M., 1987, Adv. Biochemical engineering/Biotechnology 34:57-84). Microcarrier technology provides a large amount of available cell growth area in the reactor vessel.

### BRIEF SUMMARY OF THE INVENTION

The present invention provides apparatus and devices for culturing cells. In one embodiment, the apparatus includes a container, a carrier within the container, an inflow, an outflow, and an agitation mechanism. The carrier includes an expandable surface area upon which cells are cultured.

In one embodiment, the carrier is a microsphere. In another embodiment, the carrier is a thread. In yet another embodiment, the carrier is a wafer. In another embodiment, the carrier is an agglomerate of several threads. In another embodiment, the carrier is any combination of microspheres, threads, wafers, or agglomerates of threads.

In another embodiment of an apparatus according to the present invention, the surface area of the carrier is reversibly expandable, i.e., the surface area is expanded and then reduced back to the original surface area.

The present invention also provides an apparatus in which the container an the carrier are integral with one another.

In one aspect, the reversibly expandable carrier is a tissue culture plate having a plurality of removable boundaries, which optionally are concentric boundaries, such that the surface area of the tissue culture plate is increased by removing boundaries as the surface area becomes suboptimal due to cell proliferation. The shape of the boundaries can be any regular or irregular shape, for example, square, rectangular, triangular, circular, linear, or nonlinear.

In another aspect of the invention, the boundaries are fixed on the tissue culture dish and the fixed boundary is sinuous, undulating, or zig-zag.

The present invention also provides a method for culturing cells using an apparatus according to the present invention. The method includes providing a container and a carrier adjacent to the container. The container has an inflow and an outflow and the carrier has an expandable surface area. In one embodiment of the method of the present invention, the surface area of carrier is reversibly expandable.

In another embodiment of the present invention, a device having a carrier with an expandable surface area on which to culture cells is disclosed. In one embodiment, the carrier is biodegradable. In another embodiment, the carrier is prepared from collagen.

In another embodiment of the device of the present invention, the carrier is a plurality of spheres. In another embodiment, the carrier is a tissue culture plate having removable boundaries, preferably concentric boundaries. In another embodiment, the carrier is integral with the device. In another embodiment, the carrier is a thread, a wafer, a sphere, an agglomerate of threads, or a combination thereof. In another embodiment of the device of the present invention, the carrier includes a surface area which is optimal for cell proliferation.

### BRIEF DESCRIPTION OF THE DRAWINGS

FIG. 1 is a side view of one embodiment of an apparatus according to the present invention.

FIG. 2A is a side view of a microsphere with cells adhered to its surface.

FIG. 2B is a side view of a microsphere with a maximum number of cells adhered to it.

FIG. 3A is a top view of one embodiment of a tissue culture dish having removable concentric boundaries, according to the present invention.

FIG. 3B is a top view of another embodiment of a tissue culture dish having removable concentric boundaries, according to the present invention.

FIG. 3C is a cross-sectional view of another embodiment of a tissue culture dish having removable vertical, straight boundaries, according to the present invention.

FIG. 4A is a cross-sectional view of another embodiment of a tissue culture dish having fixed, zig-zag boundaries, according to the present invention.

FIG. 4B is a cross-sectional view of another embodiment of a tissue culture dish having fixed, sinuous boundaries, according to the present invention.

FIG. 5 is a bar graph detailing the number of cells recovered from different carrier materials.

FIG. 6 is a bar graph detailing the viability of the cells recovered from different carrier materials.

### DETAILED DESCRIPTION OF THE INVENTION

The present invention described several embodiments of an apparatus for culturing cells. In each of the embodiments, the present invention includes an expandable surface area to continually culture cells over a period of time until harvest. Adherent cells have an optimal surface area upon which to attach and proliferate. If the surface area is too large or too small for the cells, then the cells will not grow. Thus, an optimal surface area for attachment and growth of cells is necessary.

In one embodiment of the present invention, with reference to the Figures, the expandable surface area may be used with a container 10 having within it a fluid 20 and at least one carrier 22 within fluid 20. Container 10 includes a fluid 20, an inflow 12 and an outflow 14. Inflow 12 and outflow 14 are in fluid and gas communication with carrier 22 through, for example, a tube 18. Cells 21 attach to expandable surface area 23 in the bioreactor on carrier 22, grow, and proliferate.

When expandable surface area 23 becomes sub-optimal for cell growth, surface area 23 is expanded, either by expanding the surface area of carrier 22 or by expanding cell culturing surface area 23, for example, which is integral with the bioreactor itself, such that surface area 23 is once again optimal for cell growth and proliferation for a larger number of cells 21.

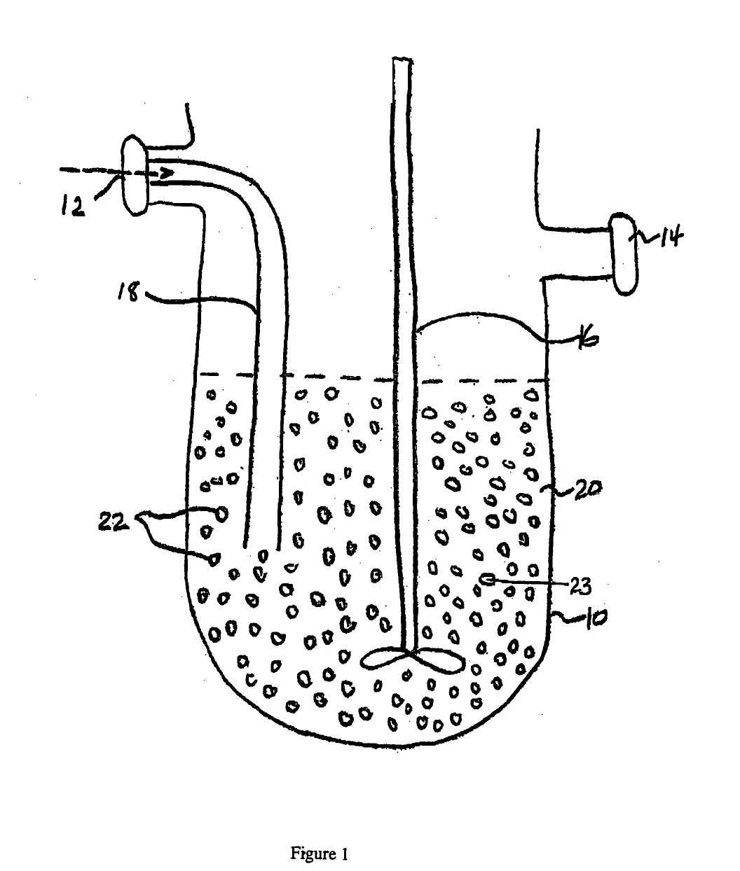

FIG. 1 depicts one embodiment of carrier 22 as microspheres. While FIG. 1 depicts a plurality of solid carriers 22 having a spherical shape, carriers 22 can be of any shape or size, and optionally have pores within them of varying size and shape. For example, carrier 22 can be a thread, an agglomerate of threads (e.g., to produce a ball form or in strand form), a wafer, a membrane, a sponge, or any combination thereof. Examples of carriers 22 are described in U.S. Patent Application No. 60/376,709, filed May 1, 2002, incorporated herein by reference in its entirety.

Carriers 22 are formed of different materials, depending for example, on the particular cells to be adhered to carrier 22, on the bioreactor design, and on the culture medium. Such materials include, but are not limited to, natural proteins such as collagen, sugar-based polymers such as dextran, plastic polymers such as polystyrene and polyhydroxybutyrate, gelatin based materials, insoluble fibers such as cellulose, hollow fibers, and ceramics, such as are described in U.S. Pat. No. 5,114,805, U.S. Pat. No. 5,830,507, and U.S. Pat. No. 6,214,618, incorporated herein by reference in their entirety. Any material which can be formed into a globular or fibrous shape is an acceptable material for carrier 22.

Materials useful for forming carriers 22 also include hyaluronic acid, starch, alginate, polyethylene glycol, and polybutyleneterephthalate. Liposome-coated materials are also useful in the present invention.

In one embodiment of the present invention, carriers 22 are commercially available microspheres, including SEPHADEX₂™ (cross-linked dextran beads) and CYTODEX™ (Sigma, St. Louis, Mo.). Other commercial suppliers of microspheres include ICN Pharmaceuticals (Costa Mesa, Calif.), Imedex (Alphretta, Ga.), Matricel (Germany), Collagen Products, Pharmacia (Piscataway, N.J.), Solo Hill Engineering (Ann Arbor, Mich.), and Verax Corporation.

Carriers 22 can be prepared from cross-linked or non-cross-linked materials. Cross-linked materials are cross-linked with any cross-linking agent, for example, glutaraldehyde, or by any cross-linking method, for example, periodic acid oxidation as described in U.S. Pat. No. 4,931,546 to Tardy, et al.

In one embodiment, shown in FIG. 1, carriers 22 are microspheres of a collagen material. Carriers 22 are resorbable or non-resorbable, and biocompatible.

In one embodiment of the invention, carrier 22 can be cultured in the presence or absence of a membrane and/or an adhesive. The membrane and adhesive can be autologous or non-autologous. The membrane and/or adhesive, for example, can be fibrin or collagen. The membrane and adhesive are non-toxic to cells 21, but the viscosity, porosity, and/or physical strength of the membrane and adhesive should be tested prior to use to determine optimal parameters for cell culture.

Membranes useful in the present invention include ChondroGide® (Ed Geistlich Sohne, Wolhusen, Switzerland), BioGide® (Ed Geistlich Sohne, Geistlich Pharma AG, Wolhusen, Switzerland), and small intestine submucosa (SIS), including the Suspend Sling™ form Mentor Corporation (Santa Barbara, Calif.), Staple Strips™ from Glycar Vascular, Inc. (Dallas, Tex.), Surgical Fabrics from Boston Scientific (Natick, Mass.), SurgiSIS™ Sling and SurgiSIS™ Mesh from Cock Biotech, Inc. (West Lafayette, Ind.), SIS Hernia Repair Device from Sentron Medical, Inc. (Cincinnati, Ohio), and the Restore® Soft Tissue Implant from DePuy Orthopaedics.

Other types of membranes useful in the present invention are disclosed in U.S. patent application Ser. No. 10/121,249, which is incorporated herein by reference in its entirety.

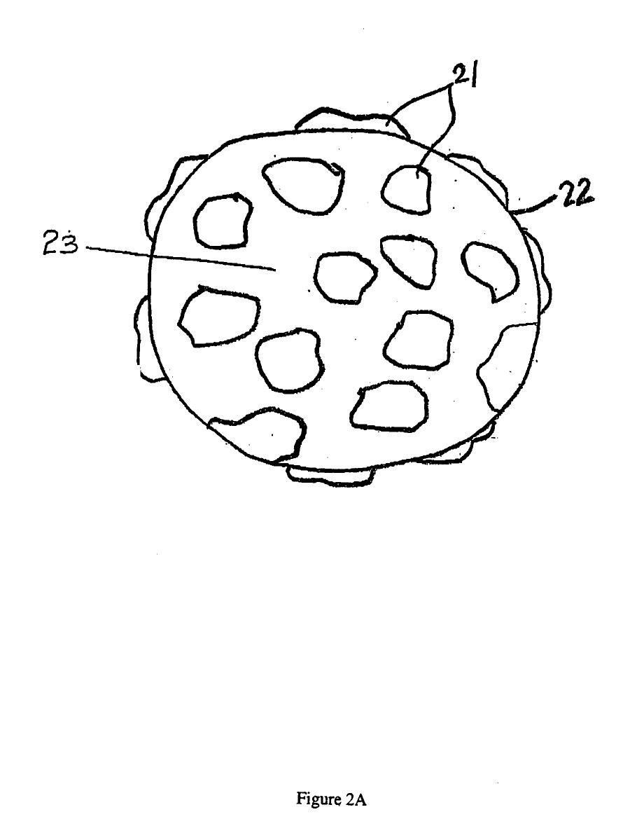

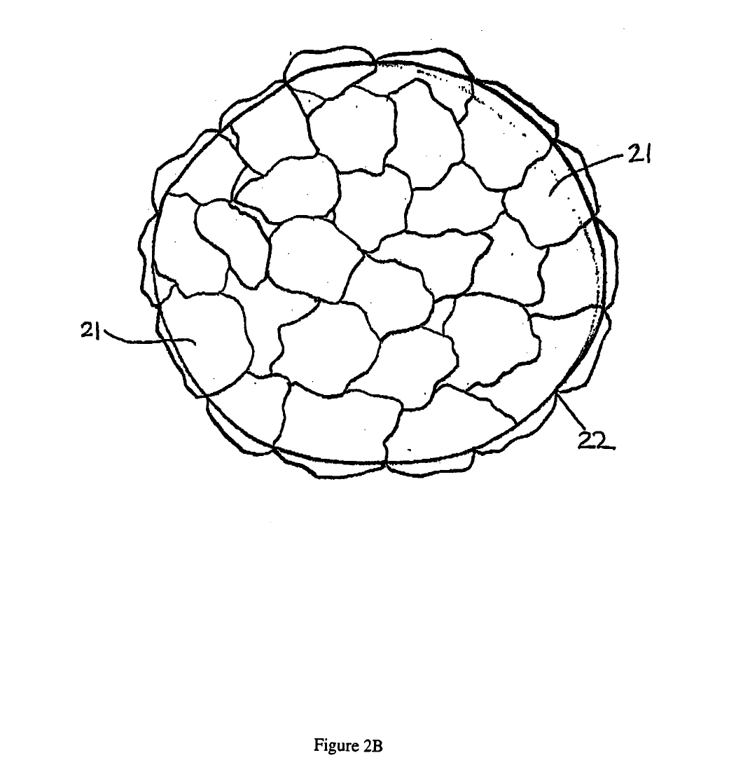

FIGS. 2A and 2B show an embodiment of the present invention in which chondrocyte cells 21 are adhered to carrier 22. The material of which carrier 22 is formed is selected to meet the requirements of and to accommodate any type of adherent cell 21. Thus, the invention is not limited to chondrocyte cells or to one particular type of carrier material. Other cells 21 including stem cells, tendon cells, neuronal cells, keratinocytes, fibroblasts, osteoblasts, myoblasts, adipocytes, hepatocytes, endocrine cells, including kidney cells, thyroid cells, suprarenal cells, and gonadal cells, and blood cells, including lymphocytes and erythrocytes, can also be cultured using the apparatuses and methods according to the present invention.

In one embodiment, the present invention includes a bioreactor having carrier 22 and an expandable surface area upon which cells attach and proliferate for cell growth. According to the present invention, the surface area of the bioreactor is expanded in a number of ways. In one embodiment according to the present invention, additional carriers 22 are added to container 10 as usable surface upon which cells 21 adhere, grow, and proliferate when surface area becomes suboptimal for cells 21. In one embodiment of the present invention, additional surface area is obtained by adding carriers 22 to container 10, when the carriers are of the same size and shape as the carriers 22 already present in container 10. In alternative embodiments, added carriers 22 are larger, smaller, or of varying sizes and shapes as compared to carriers 22 already in container 10.

Cell density varies in relation to the size of carriers 22, and it also depends on the phase of cell growth. Thus, obtaining an optimum ratio of cells to expandable growth area depends in part upon the phase of cell growth and cell density. Carriers 22 range in size from about 20 microns to about 500 microns. In one embodiment, carriers 22 that are wafers may range in diameter from about 20 microns to about 2 millimeters.

In one embodiment of the present invention, surface area is increased by hydrating carrier 22 with a hydrating agent, such as water, fatty acids, oils and liposomes. Carriers 22 can be completely dry when added to the bioreactor apparatus, or at any stage of hydration, such that further hydration causes an increase in surface area.

In any one of the above embodiments, optimal surface area is preferably determined prior to adding more carriers 22. This calculation is determined by preliminary experiments with the particular carrier material and type of cell. After preliminary experiments are performed, specific amounts of carriers 22 are added to the bioreactor apparatus, the amounts being measured for example, by counting the carriers, adding a particular weight of carriers 22, and/or adding a particular surface area (for example, for a round microsphere, the formula would be ⅓ πr³). Expanding the surface area in this way can be repeated a number of times, up to about 20 repetitions. Preferably, the cells are harvested after 2 to 10 repetitions, and more preferably, the cells are harvested after 2 to 5 repetitions.

In another embodiment of the invention, the expandable surface area is increased by dissolving or breaking down carrier 22 with an enzyme or other solution which will dissolve or degrade carrier 22 but which will not affect cell 21. In one embodiment, after carriers 22 are dissolved, cells 21 are pelleted, harvested, washed, and re-suspended with fresh carriers 22 to continue cell growth and proliferation. In one embodiment of the present invention, fresh carriers 22 are larger than carriers 22 previously in the bioreactor. In a separate embodiment, fresh carriers 22 are smaller than carriers 22 previously in the bioreactor. In yet another embodiment, fresh carriers 22 vary in size from small to large carriers 22 are compared with carriers 22 previously in the bioreactor. In any one of the above three embodiments, optimal surface area is determined prior to addition of fresh carriers 22. This calculation is performed by resuspending cells 21 in fluid 20, counting the number of cells 21 present in fluid 20, and determining the optimal surface area on which these cells will grow. This process can be repeated several times, as described above.

For any of the embodiments discussed above, container 10 can be, e.g., a glass, ceramic, or plastic material. In one embodiment, container 10 is a plastic polymer, including, but not limited to polystyrene, polyethylene, PVC, polystyrene, and other shapeable artificial resins.

For any one of the embodiments discussed above, agitation mechanism 16 is preferably a stirring mechanism, for example, a magnetic stirring bar or a motor-driven stirring rod as depicted in FIG. 1. Agitation can also occur through a shaking mechanism, a rolling mechanism, or by cyclic flow.

In another embodiment of the present invention, carrier 22 is a tissue culture plate 25 having an inflow 12 and an outflow 14. Tissue culture plate 25 can be of any surface area size and having an expandable and contractable surface area. Factors to consider when choosing the surface area size of tissue culture plate 25 include temperature, carbon-dioxide content, gas exchange, cell culture medium, whether the tissue culture dish is coated, amount of cell culture medium to cells, and whether the cells have been pretreated or preincubated.

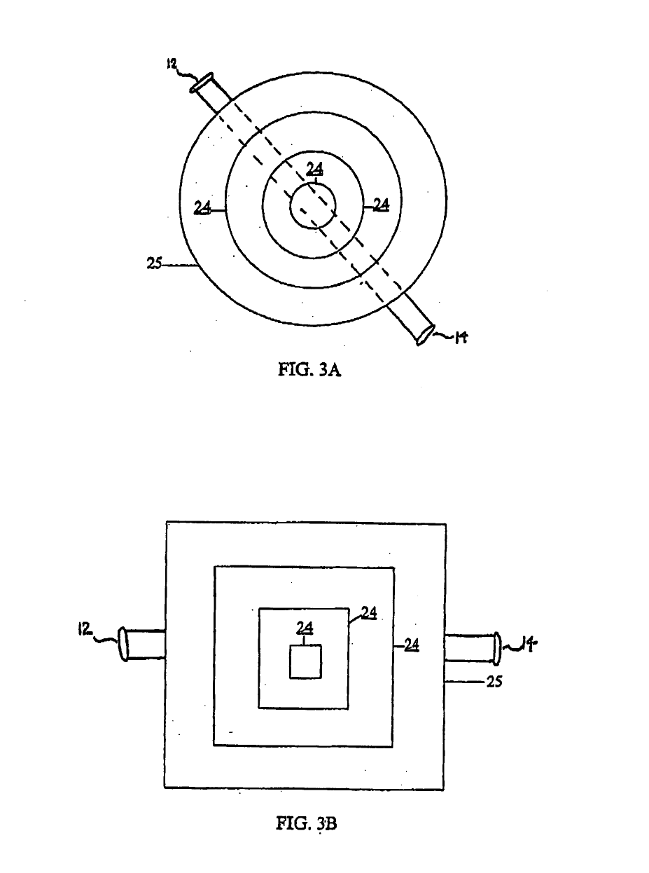

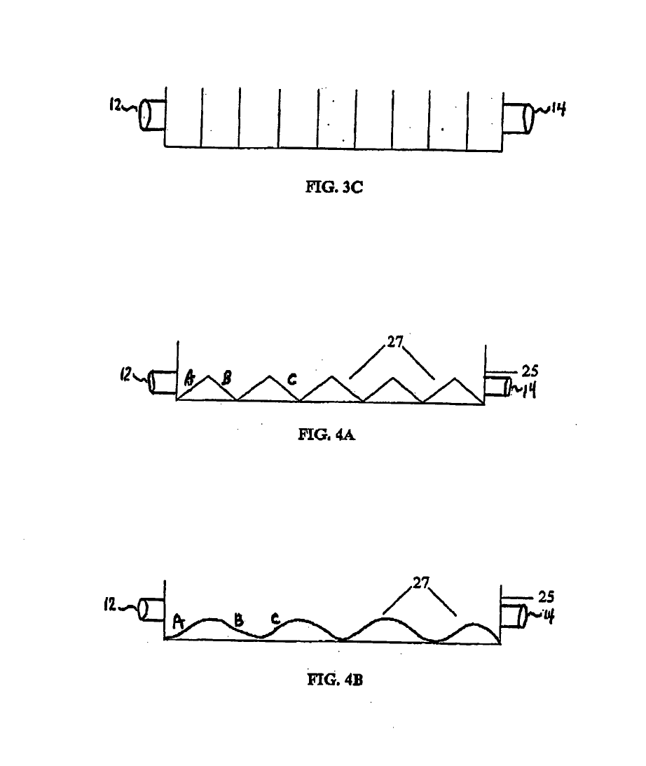

In one embodiment, tissue culture plate 25 has removable boundaries 24, as shown in FIGS. 3A and 3B, that allow the surface area of the bioreactor to be adjusted through the cell culture process. FIG. 3A demonstrates tissue culture plate 25 in a round shape, having removable boundaries 24. FIG. 3B demonstrates tissue culture plate 25 in a square shape. The shape of tissue culture plate 25 is not essential to the invention. Thus, tissue culture plate 25 can be of any regular or irregular shape, including, but not limited to round, square, triangular, rectangular, and rhomboid.

In one embodiment of the present invention, removable boundaries 24 are concentric, i.e., the boundaries have the same center. In one embodiment as shown in FIGS. 3A and 3B, cells 21 are seeded in the innermost boundary of tissue culture plate 25, and adjacent outer boundaries 24 are removed as surface area within the boundary becomes sub-optimal for cell growth. Generally, surface area becomes sub-optimal for cell growth at about 65 percent to 90 percent cell confluence. At this point, the cells are either harvested or a boundary 24 is removed. In another embodiment, cells 21 are seeded in the outermost boundary of tissue culture plate 25, and adjacent inner boundaries 24 are removed as surface area within the boundary becomes sub-optimal for cell growth.

In another embodiment of the present invention, consecutive removable vertical boundaries 24 may be used, as depicted in FIG. 3C. In this embodiment, cells 21 are seeded in any one of the bounded areas, and one or more adjacent boundaries 24 are removed, as above, to increase the area as surface area becomes suboptimal.

In yet another embodiment of the present invention, tissue culture plate 25 has fixed, undulating boundaries 27, as depicted in FIGS. 4A and 4B. FIG. 4A depicts boundaries 27 as zig-zag boundaries. The angle under each peak of undulating boundary 27 is the same or may be varied, and is an angle between 0 and 180 degrees. Cells 21 are seeded at any point on the fixed boundary and continually grow along an edge of the tissue culture plate 25, for example, on section A, section B, etc.

Another embodiment of the present invention is depicted in FIG. 4B, wherein fixed boundaries 27 are continuous or variable, for example, undulating or sinuous curves. In one embodiment, the peaks and valleys of these boundaries are rounded, and the area under each peak of undulating boundary 27 is the same.

In one embodiment of the present invention, tissue culture plate 25 and removable boundaries 24 or fixed boundaries 27 are formed of the same material. Material suitable for the bioreactor include, but are not limited to a plastic polymer, or a combination of plastic polymers, including, but not limited to polystyrene, polyethylene, polypropylene, glass, PVC, and other non-toxic, sterilizable plastic materials.

Alternatively, tissue culture dish 25 and removable boundaries 24 or fixed boundaries 27 are of different materials. In one embodiment, tissue culture dish 25 is a plastic polymer, such as polystyrene, and removable boundaries 24 or fixed boundaries 27 are formed of a material on which cells 21 do not adhere, for example, glass, teflon, siliconized surfaces, and negatively charged surfaces.

In another embodiment of the present invention, carriers 22 and tissue culture dish 25 are used together to culture cells. Tissue culture dish 25 can be used as a container for the bioreactor apparatus and carriers 22 and/or culture dish 25 provide an expandable surface area upon which cells 21 can proliferate.

Typically, all of the embodiments discussed above have one or more common features. Fluid 20, in an embodiment of the present invention, is a cell culture medium. Cell culture medium is different for different cell types, and the same cell type can be cultured using different types of medium. Similarly, cell culture media can be supplemented with various nutrients, autologous materials including blood serum, growth factors, antibiotics, anti-fungals, sugars, etc.

By way of example, in one embodiment of the invention in which cells 21 are chondrocytes, fluid 20 is chondrocyte cell culture medium containing 2.5 milliliters of gentamycin sulfate an initial concentration of 70 micromole/liter, 4.0 milliliters of amphotericin at an initial concentration of 2.2 micromole/liter (tradename Fungizone®, an antifungal available from Squibb), 15 milliliters 1-ascorbic acid at an initial concentration of 300 micromole/liter, 100 milliliters of fetal calf serum to a final concentration of 20%, and the remainder DMEM/F12 media to produce about 500 milliliters of growth media.

Fluid 20 may also contain nutrients for optimal cell growth. It is likely that large-scale production of certain cells 21, such as cells that are difficult to culture, like tumor cells or genetically altered cells lacking genes normally found in that cell type, may be desirable. Thus, other fluids, including, but not limited to specialized media for producing a specific phenotype or genotype of cell 21 that is not the optimal phenotype or genotype, are also included in the invention.

Sterile conditions and optimal growth parameter are also important for cell growth and proliferation. In one embodiment of the present invention, the bioreactor apparatuses are of a relatively small size, and are workable both under a laminar flow hood and in a centrifuge. The apparatuses are also easily manipulated in an incubator. Gas exchange occurs via inlet 12 (FIG. 1), and can also occur via diffusion or via stirring mechanism 16 (FIG. 1). In an embodiment of the present invention, cells 21 and carriers 22 are added to the bioreactor chamber via inlet 12. Also, in a preferred embodiment of the invention, the bioreactor apparatuses include a sterile, preferably screwable, cap.

In all of the above embodiments, cells may be harvested along any step in the cell culture process, depending on the stage of development of the cells and the eventual use of the cells. Generally, cells are harvested when a confluency of about 65 percent to about 95 percent is reached. Criteria for determining when cells should be harvested include counting cells per aliquot of cell culture medium to determine confluency.

For any one of the tissue culture embodiments, cells are harvested as they approach between about 65 percent and 95 percent confluence of the entire culturing surface area.

These and other aspects of the instant invention may be better understood from the following example, which are meant to illustrate, but not to limit, the present invention.

### EXAMPLE 1

### Cell Seeding on Collagen Microspheres

One hundred thousand human chondrocyte cells in 100 microliters of cell culture medium were seeded into a well of a six-well tissue culture plate for use as a control cell population. Three batches of forty milligrams of collagen microspheres (Mcox-001; Imedex, France) were washed two times with phosphate buffered saline (PBS). Each batch was seeded with 100,000 human chondrocytes per forty milligrams of collagen microspheres. One hour after seeding, 3.5 milliliters of cell culture medium were added, and the spheres were incubated on a shaker for three days at 37 degrees Celsius.

After the three-day incubation, an additional 10, 20, or 40 milligrams of microspheres were added, respectively, to be 1, 2 and 3. One week after initial seeding of the microspheres, the medium being changed once within this period, an additional 10, 20, or 40 milligrams of fresh microspheres were added, respectively, to batches 1, 2, and 3.

After 8 days, the microspheres were digested with collagenase and cells were typsinized. Cell number and viability were determined. Table 1 shows cell number and viability before and after seeding.

TABLE 1

| | |
|---|---|
| Pre-seeding | Eight days Post-Seeding |

| | Weight of | Number of | Number of | Percent |
|---|---|---|---|---|
| | Collagen | Cells | Cells | Viability |
| Control | — | 100,000 | — | — |
| Batch 1 | 3 mg | 100,000 | 157,500 | 95% |
| Batch 2 | 3 mg | 100,000 | 347,500 | 98% |
| Batch 3 | 3 mg | 100,000 | 325,000 | 98% |

From these data, it can be concluded that cell number and viability can be propagated with the addition of fresh microspheres periodically throughout the culturing process, until a desired maximum number of cells is achieved. This can be accomplished without having to culture cells in the traditional manner, i.e., using a finite surface area.

### EXAMPLE 2

### Comparison of Different Carriers and Materials

The goal of this example is to demonstrate the differences between different types of carrier materials, namely collagen threads crosslinked with and without glutaraldehyde, collagen microspheres, and collagen membranes, each used alone.

The Chondro-Gide® membrane was used as a positive and a negative control for this experiment. Chondrocyte cells without carrier material was also used as an additional control. The test materials were as follows:
Threads pressed into a globular shape with a diameter of about 0.5 centimeters and cross-linked with glutaraldehyde (Pcox-R1; Imedex);
Threads pressed into a globular shape with a diameter of about 0.5 centimeters and cross-linked without glutaraldehyde (Pcox-001; Imedex; see cross-linking method in Tardy, et al. U.S. Pat No. 4,931,546);
Microspheres cross-linked without glutaraldehyde (Mcox-001; Imedex); and
bi-layered CR-1 membrane (Imedex), cross-linked with glutaraldehyde.

Five million frozen human chondrocytes were thawed, washed with PBS, and cell viability was determined. After thawing and washing, 4.2×10⁶ cells were present in solution, and of them, 62% were viable. The cells were then split into two flasks, incubated for three days at 37 degrees Celsius, and passaged in the traditional way to obtain 4.3×10⁶ and 5×10⁶ cells, respectively. Viability for each of the flasks was 89% and 86%, respectively. The cell suspensions were then combined, for a total of 9.3×10⁶ cells, and a mean viability of 87.5%.

Each of the collagen materials was washed with PBS, and added to one well each on a 12-well tissue culture plate. The chondrocyte suspension was then typsinized, and a suspension having 100,000 cells was applied to each of the wells containing the prepared carriers material. The cells plus carrier material mixture was incubated for three days at 37 degrees Celsius.

Control cells were harvested by first trypsinizing the cells, then washing and pelleting the cells by centrifugation. Cell quantity and viability were determined. Cells seeded on the collagen materials were treated with trypsin and collagenase, and dissolution of the collagen materials were periodically monitored microscopically. After dissolution of the collagen carrier, the cells were centrifuged and counted.

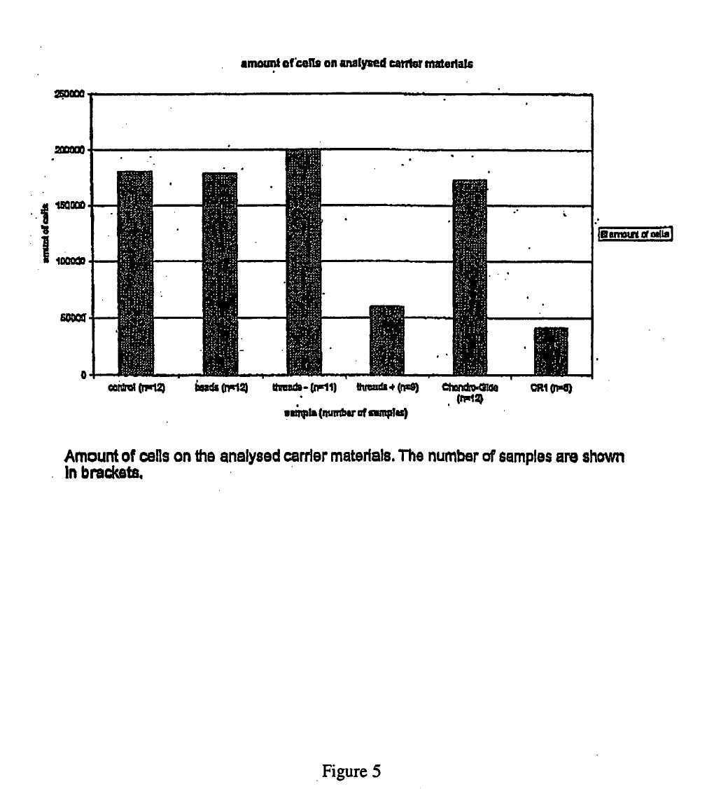

From the control group (n=12) and the sphere group (n=12), approximately 175,000 cells were harvested after three days incubation. Threads cross-linked without glutaraldehyde n=11) yielded approximately 200,000 cells, while threads cross-linked with glutaraldehyde (n=9) yielded only about 55,000 cells. It was noted that the threads cross-linked with glutaraldehyde did not dissolve easily in the collagenase, and the cells had to be collected from the intact threads. Approximately 175,000 cells were collected from the Chondro-Gide® (n=12). Finally, the CR-1 membrane (n=6) yielded approximately 40,000 cells (See FIG. 5).

All of the spheres, threads cross-linked without glutaraldehyde, and Chondro-Gide® provided positive results, performing at least as well as the control group. The threads cross-linked with glutaraldehyde and the CR-1 membrane yielded substantially less than the control group; however, the lower yield may be attributed to a number of different factors, including the strength with which the cells bound the carrier material. The stronger the cells bind the carrier, the less likely it is that the bound cells will be counted toward yield.

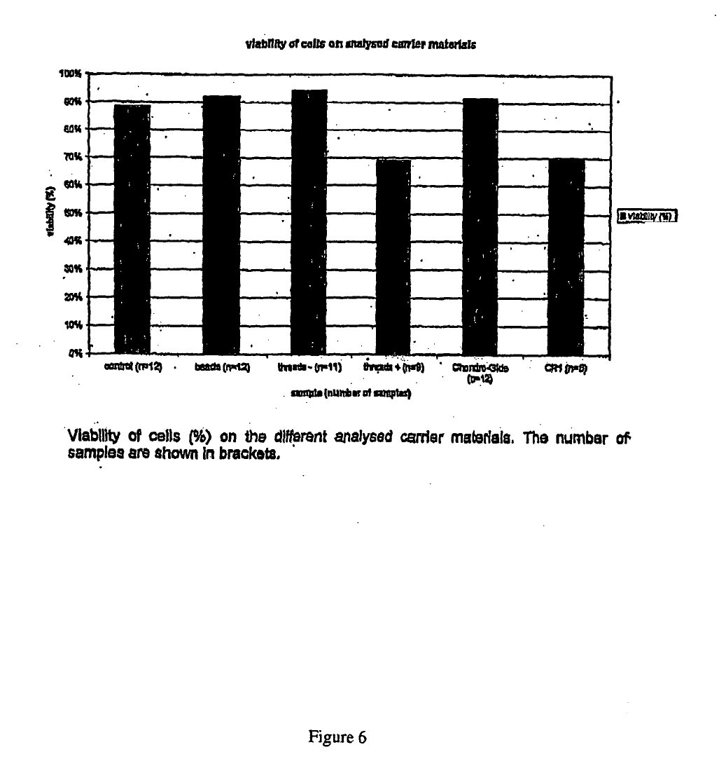

Viability of the cells was also assessed in each group. FIG. 6 is a bar graph showing the viability of the cells as compared with the control sample. All of the groups exhibited greater than 90% viability, with the exception of the threads cross-linked with glutaraldehyde and the CR-1 membrane, both of which yielded about 70% viability. The lower viability may be attributed to the fact that these carriers were digested for an extended time with collagenase, as indicated by an increase in viability when the carrier materials were mechanically reduced to smaller pieces (data not shown).

The disclosures of each and every patent, patent application, and publication cited herein are hereby incorporated by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An apparatus for culturing cells comprising:
(a) a container;
(b) a carrier with said container, wherein said carrier includes an expandable surface area upon which cells are cultured;
(c) an inlet fluidly communicating with said surface area; and
(d) an outlet fluidly communicating with said surface area.

2. The apparatus according to claim 1, wherein said carrier is integral with said container.

3. The apparatus according to claim 2, wherein said carrier is a portion of said container.

4. The apparatus according to claim 1, wherein said surface area is reversibly expandable.

5. The apparatus according to claim 4, wherein said carrier is a biodegradable material.

6. The apparatus according to claim 5, wherein said biodegradable material is collagen.

7. The apparatus according to claim 1, wherein said carrier is a biodegradable material.

8. The apparatus according to claim 1, wherein said surface area includes an optimum surface area upon which cells will proliferate.

9. The apparatus according to claim 1, wherein said carrier is selected from the group consisting of a thread, a wafer, a sphere, a membrane, or any combination thereof.

10. The apparatus according to claim 9, wherein said carrier is a sphere.

11. The apparatus according to claim 10, wherein said carrier is a plurality of spheres.

12. The apparatus according to claim 1, wherein said carrier includes a tissue culture plate having removable boundaries.

13. The apparatus according to claim 12, wherein said boundaries are concentric.

14. The apparatus according to claim 1, wherein said carrier includes a tissue culture plate having fixed boundaries.

15. An apparatus for culturing cells comprising a carrier, said carrier having an expandable surface area upon which cells are cultured.

16. The apparatus according to claim 15, wherein said surface area is reversibly expandable.

17. The apparatus according to claim 15, wherein said carrier includes a tissue culture plate having removable boundaries.

18. The apparatus according to claim 17, wherein said boundaries are concentric.

19. A method for culturing cells comprising:
(a) providing a container and a carrier adjacent the contain, wherein the container includes an inflow and an outflow, and wherein the carrier includes an expandable surface area;
(b) supplying cells through an inflow to the carrier, wherein said cells will adhere to the carrier and proliferate;
(c) continually supplying nutrients through the inflow of the container.

20. The method of claim 19, wherein said surface area of the carrier is reversibly expandable.

21. The method of claim 19, wherein said carrier is spherical.

22. The method according to claim 19, wherein said carrier is a plurality of spheres.

23. The method according to claim 19, wherein said carrier includes a tissue culture plate having removable boundaries.

24. The method according to claim 23, wherein said boundaries are concentric.

25. The method according to claim 19, wherein said carrier is a biodegradable material.

26. The method according to claim 25, wherein said biodegradable material is collagen.

27. The method according to claim 19, wherein said carrier is integral with said container.

28. The method according to claim 19, wherein said carrier is a portion of said container.

29. A device for culturing cells comprising an carrier including an expandable surface area upon which cells are cultured.

30. The device of claim 29, wherein said carrier is a biodegradable material.

31. The device of claim 29, wherein said biodegradable material is collagen.

32. The device of claim 29, wherein said surface area includes an optimum surface area upon which cells will proliferate.

33. The device of claim 29, wherein said carrier is selected from the group consisting of thread, a wafer, a sphere, a membrane, and any combination thereof.

34. The device of claim 29, wherein said carrier is spherical.

35. The device of claim 29, wherein said carrier is a plurality of spheres.
